(19) United States
(12) Patent Application Publication
(10) Pub. No.: US 2023/0107876 A1
(43) Pub. Date: Apr. 6, 2023

(54) Disposable Flow Through Diagnostic Device and Method of Construction Thereof

(71) Applicant: Creganna Unlimited Company, Ballybrit (IE)

(72) Inventors: David W. Wright, Littleton, CO (US); Fred Mitchell, Windsor, CO (US); Benjamin A. Castillo, Highlands Ranch, CO (US)

(73) Assignee: Creganna Unlimited Company, Ballybrit (IE)

(21) Appl. No.: 17/854,734

(22) Filed: Jun. 30, 2022

Related U.S. Application Data
(60) Provisional application No. 63/218,250, filed on Jul. 2, 2021.

(51) Int. Cl. B01L 3/00

(52) U.S. Cl. CPC B01L 3/502715; B01L 2200/026; B01L 2200/0689; B01L 2200/16; B01L 2300/0816; B01L 2400/0481; B01L 2300/0877; B01L 2300/0861

(57) ABSTRACT

A disposable diagnostic device includes a body having a first channel and a second channel spaced from the first channel. A shroud is operably fixed to the body and encloses a chamber which is configured in a hermetically sealed-off relation from the first and second channels when the device is in a non-activated first state and is in open communication with at least one of the first and second channels when the device is in an activated second state. A reactant and an inert gas are disposed in the chamber such that the inert gas protects the reactant from being exposed to contaminants when the device is in said non-activated first state. A method of constructing a disposable diagnostic device is also disclosed.

### CROSS-REFERENCE TO RELATED APPLICATIONS

This application claims priority from U.S. Application Ser. No. 63/218,250, filed Jul. 2, 2022, the disclosure of which is incorporated herein by reference.

### BACKGROUND

### 1. Technical Field

This invention relates generally to in-vitro diagnostics, and more particularly to disposable diagnostic cartridges containing labile contents in a compartment thereof and to methods constructing such disposable diagnostic cartridge having labile contents therein.

### 2. Related Art

Diagnostic tests are increasingly being used to determine the state or condition of a biological environment, such as in human healthcare, agriculture, livestock management, municipal systems management, and national defense, by way of example and without limitation. A new market is emerging wherein diagnostic tests are being performed at the point-of-care. The diagnostic test can be complex, requiring multiple reagents and multiple steps to execute an assay. An assay is a sequence of steps or procedures used to measure the presence or absence of a substance in a sample, the amount of a substance in a sample, or the characteristics of a sample. An example of a common and relatively simple point-of-care assay, which can be readily conducted by a layperson, is a blood glucose test. In this test, generally speaking, the blood is mixed with glucose oxidase, which reacts with the glucose in the sample, creating gluconic acid, wherein the gluconic acid reacts with a chemical, typically ferricyanide, producing ferrocyanide. Current is passed through the ferrocyanide and the impedance reflects the amount of glucose present.

Although the aforementioned blood glucose assay is relatively common and simple, many assays are far more complex in that they require highly valuable, labile reactants, sensitive to moisture and/or gasses that are generally present in an ambient environment, such as oxygen, to be mixed with a fluid or gas reagent to perform the test and provide the desired quantitative test results. Some known labile reactants include lyophilized reagents and gases, such as CO₂ incorporating a radioactive carbon isotope, for example. Given the labile nature of such reactants, it is critical to ensure such selected reactant(s) is protected against exposure to surrounding contaminants prior to use and during use to avoid causing a breakdown of the reactant, which ultimately results in compromising the results of the assay.

To avoid breakdown of labile reactant(s) contained in known diagnostic test apparatus, it is a common to incorporate the labile reactant into the diagnostic test apparatus in a dry-room environment. Although dry-room environments, typically controlled having a 15% moisture content or less, are useful, personnel working in dry-room environments are commonly required to use breathing apparatus to avoid harm to their lungs. As such, operating, maintaining and working in a dry-room environment is cumbersome and costly.

In addition to having to assemble the aforementioned diagnostic test apparatus in a dry-room environment, it is common to package the assembled diagnostic test apparatus in a sealed package having a controlled internal environment within the sealed package, typically controlled via the incorporation of a desiccant to prevent moisture within the sealed package and surrounding environment from causing the labile reactant to break down. If such precautions in packaging the diagnostic test apparatus are not taken, the risk of the labile reactant being broken-down is greatly increased. Even when such packaging precautions are taken, the viable shelf life of the diagnostic test apparatus can be relative short, such as weeks, particularly in a thermally and/or moisture content uncontrolled environment.

### SUMMARY OF THE INVENTION

In accordance with one object of the invention, a single-use, consumable diagnostic cartridge is provided that addresses at least those problems discussed above with regard to known single-use, consumable diagnostic cartridges.

In accordance with an aspect of the invention, a disposable diagnostic device is provided. The disposable diagnostic device includes a body having a first channel and a second channel spaced from the first channel. A shroud is operably fixed to the body. The shroud encloses a chamber, wherein the chamber is configured in hermetically sealed-off relation from the first channel and the second channel when the disposable diagnostic device is in a non-activated first state. The chamber is configured for open communication with at least one of the first channel and the second channel when the disposable diagnostic device is in an activated second state. A reactant is disposed in the chamber, and an inert gas is disposed in the chamber. The inert gas protects the reactant from being exposed to contaminants while in the chamber when the disposable diagnostic device is in the non-activated first state.

In accordance with another aspect, the first channel is sealed-off from said chamber by a first rupturable member covering a first port when said first rupturable member is in a non-ruptured state and said first channel is in fluid communication with said chamber through said first port when said first rupturable member is in a ruptured state, said second channel is sealed-off from said chamber by a second rupturable member covering a second port when said second rupturable member is in a non-ruptured state and said second channel is in fluid communication with said chamber through said second port when said second rupturable member is in a ruptured state.

In accordance with another aspect, first rupturable member is fixed to said body and wherein said second rupturable member is fixed to said body.

In accordance with another aspect, first rupturable member and said second rupturable member are a single piece of material.

In accordance with another aspect, further including an agitation mechanism disposed within said chamber, said agitation mechanism being configured to agitate the flow of a media entering said chamber from one of said first channel through said first port and said second channel through said second port to mix said media with said reactant.

In accordance with another aspect, agitation mechanism is formed on an inner surface of said shroud, said inner surface being exposed to said chamber.

In accordance with another aspect, inner surface has a plurality of protrusions extending into said chamber to form said agitation mechanism.

In accordance with another aspect, agitation mechanism is formed by one or more solid members contained in said chamber, said one or more solid members being free to move within said chamber when at least one of said first and second rupturable members is in said ruptured state to mix said media with said reactant.

In accordance with another aspect, one or more solid members is restrained against movement within said chamber when said first and second rupturable members are in said non-ruptured states.

In accordance with another aspect, an agitation mechanism is formed by ferrous material contained within said chamber, said ferrous material being configured to move within said chamber upon being selectively exposed to an external magnetic field.

In accordance with another aspect, shroud is formed of a compliant material configured to be depressed and substantially flattened to motivate a flow of a media entering said chamber from one of said first channel through said first port and from said second channel through said second port and out the other of said first port through said first channel and out said second port through said second channel.

In accordance with another aspect, shroud has a compressed state defining a first volume inside said chamber when said first rupturable member is in its non-ruptured state and when said second rupturable member is in its non-ruptured state, and an expanded state defining a second volume inside said chamber when at least one of said first rupturable member is in its ruptured state and when said second rupturable member is in its ruptured state, said second volume being greater than said first volume.

In accordance with another aspect, a method of constructing a disposable diagnostic device is provided. The method includes: providing a diagnostic cartridge body having a plurality of microfluidic channels; providing a shroud; disposing a reactant between the shroud and the diagnostic cartridge body; fixing the shroud to the diagnostic cartridge body to seal-off a chamber between the shroud and the diagnostic cartridge body, wherein the reactant is contained in the chamber and the chamber is configured for selective fluid communication with the plurality of microfluidic channels; and performing the disposing and fixing steps in a vacuum atmosphere.

In accordance with another aspect, the method can further include providing an inert gas in the vacuum atmosphere and sealing some of the inert gas in the chamber with the reactant.

In accordance with another aspect, the method further includes performing the fixing step without a dry-room atmosphere.

In accordance with another aspect, the method can further include providing an agitation mechanism in the chamber to facilitate mixing the reactant with a reagent.

### BRIEF DESCRIPTION OF THE DRAWINGS

These and other aspects, features and advantages of the invention will become more readily appreciated when considered in connection with the following detailed description of presently preferred embodiments and best mode, appended claims and accompanying drawings, in which:

FIG. 1 is an isometric view of a disposable diagnostic device constructed in accordance with one aspect of the invention;

FIG. 2 is cross-sectional view of the disposable diagnostic device taken generally along the line 2-2 of FIG. 1;

FIG. 3 is a similar view to FIG. 2 of the disposable diagnostic device showing the disposable diagnostic device upon being actuated;

FIGS. 3a and 3b are enlarged views of the encircled regions 3a and 3b of FIG. 3, wherein FIG. 3b illustrates an outlet and FIG. 3a illustrates an inlet, with the enlarged actuated illustration of FIG. 3a being similar to that as shown for FIG. 3a;

FIG. 4 is a view similar to FIG. 1 of a disposable diagnostic device constructed in accordance with another aspect of the invention;

FIG. 5 is cross-sectional view of the disposable diagnostic device of FIG. 4 taken generally along the line 5-5;

FIG. 5a is cross-sectional view similar to FIG. 5 of a disposable diagnostic device in accordance with another aspect of the invention;

FIG. 5b is a similar view to FIG. 5a of the disposable diagnostic device showing the disposable diagnostic device of FIG. 5a upon being partially actuated;

FIG. 6 is a similar view to FIG. 5 of the disposable diagnostic device showing the disposable diagnostic device of FIG. 4 upon being actuated;

FIG. 7 is a view similar to FIG. 1 of a shroud assembly of a disposable diagnostic device constructed in accordance with another aspect of the invention; and

FIG. 8 is cross-sectional view of a disposable diagnostic device having the shroud assembly of FIG. 7 assembled thereto taken generally along the line 8-8.

### DETAILED DESCRIPTION OF PRESENTLY PREFERRED EMBODIMENTS

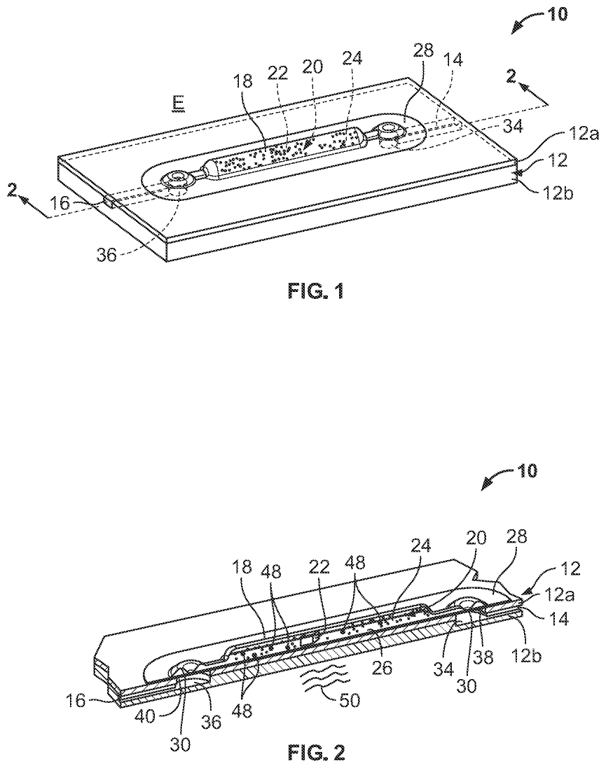

Referring in more detail to the drawings, FIG. 1 illustrates a disposable diagnostic device, also referred to as diagnostic cartridge, and referred to hereafter as cartridge 10, constructed in accordance with one aspect of the invention for use in performing an assay in a qualitative and quantitative analysis on a specimen. The cartridge 10 includes a body 12 having a first microfluidic channel for conveying a fluid and/or gas, referred to hereafter as first channel 14, and a second microfluidic channel for conveying a fluid and/or gas, referred to hereafter as second channel 16, with first channel 14 being spaced from second channel 16 by a storage chamber, which can also function as a mixing chamber, and referred to hereafter simply as chamber 20. The body 12 can be constructed, if desired, having an upper body portion 12a and a lower body portion 12b fixed to one another to facilitate forming the desired contour and path(s) of the channels therein, shown by way of example as first channel 14 and second channel 16 be formed in lower body portion 12b and enclosed by upper body portion 12a. A blister, also referred to as shroud 18, is operably fixed to a generally planar surface of upper body portion 12a. The shroud 18 encloses the chamber 20, wherein the chamber 20 is configured in hermetically sealed-off relation from the first channel 14 and the second channel 16 when the disposable diagnostic device 10 is in a non-actuated state, also referred to as non-activated first state. The chamber 20 is configured for open communication with at least one of the first channel 14 and the second channel 16, meaning that fluid and/or gas is free to flow therebetween, when the disposable diagnostic device 10 is in an actively deployed, actuated state, also referred to as activated second state. A reactant 22, such as a labile reactant, e.g. biological enzymes, lyophilized reagents including nucleic acid sequences, radioactive carbon isotopes, and any other form of highly sensitive biologic material, or otherwise, is disposed in the chamber 20 along with an inert gas 24. The inert gas 24, such as argon, by way of example and without limitation, occupies the volume within the entirety of the chamber 20 and protects the labile reactant 22 from being exposed to fluid, solid and/or gas contaminants, e.g. oxygen, while in the chamber 20 when the disposable diagnostic device 10 is in the non-activated first state. Accordingly, the reactant 22 is protected against being unintentionally broken down and degraded while stored in the cartridge 10, as intended, for future use in conducting an assay. With the chamber 20 being hermetically sealed off from the first channel 14, the second channel 16 and surrounding environment E by the shroud 18, the cartridge 10 is well suited for shipment and storage over an extended period of time, such as years, without need of additional protective features, including desiccants, or the need for specialized packaging.

The cartridge 10 is readily manufactured without need of a dry-room environment, and thus, the total cost (including time, labor, space, capital equipment, etc.) of manufacture is significantly reduced in comparison to a cartridge requiring use of a dry-room environment for manufacture. In accordance with one aspect, the cartridge 10 is manufactured under a vacuum atmosphere, such that the labile reactant 22 is protected from exposure to moisture and other forms of contamination, such as from oxygen, while being disposed and hermetically sealed in the chamber 20. In accordance with one aspect, the labile reactant 22 can be disposed into the chamber 20 prior to fixing the shroud 18 to the body 12, wherein the inert gas 24 occupies the vacuum atmosphere and is sealed with the chamber 20 along with the labile reactant 22 upon fixing the shroud 18 to the body 12.

The shroud 18 can be formed of any suitable flexible, compliant material or materials to bound and encapsulate the chamber 20 of a predetermined volume. The shroud 18, as shown by way of example and without limitation, includes a bottom surface or layer 26, formed without any predefined rupturable or frangible valve, opening or otherwise, and an upper layer 28. Though the bottom surface 26 is described as being valve or opening free, it is contemplated that a predefined valve or opening could be formed in the bottom surface 26, if desired, though not necessary as a result of upper layer 28 having a plurality of opening, piercing or puncture members 30 configured to form openings 32 in the bottom layer 26 over first and second ports 34, 36 of first and second channels 14, 16, respectively. The upper layer 28 can be formed of the same type of material as the bottom layer 26, or from a different type of material, as desired. The upper layer 28 is sufficiently sized to allow the fluid/gas disposed therein to create a bulbous, expanded portion bounding the reservoir chamber 20, wherein the upper layer 28 is flexible and tough, thereby allowing the bulbous portion to be depressed and actuated upon if desired to facilitate motivating flow of the fluid/gas through one of the first and second channels 14, 16. The bottom and upper layers 26, 28 can be bonded to one another about their respective out peripheries via any suitable bonding process upon disposing the labile reactant 22 and inert gas 24 therebetween, such as a suitable welding or adhering process.

The first channel 14 is sealed-off from the chamber 20 by a first valve, also referred to as first rupturable member 38 covering the first port 34 when the first rupturable member 38 is in a non-ruptured state. The first channel 14 is in fluid communication with the chamber 20 through the first port 34 when the first rupturable member 38 is in a ruptured state. The second channel 16 is sealed-off from the chamber 20 by a second rupturable member 40 covering the second port 36 when the second rupturable member 40 is in a non-ruptured state. The second channel 16 is in fluid communication with the chamber 20 through the second port 36 when the second rupturable member 40 is in a ruptured state. The first and second rupturable members 38, 40 can be selectively ruptured concurrently with one another, or separately from one another at different times, as desired.

The first rupturable member 38 is fixed to the body 12 and the second rupturable member 40 is fixed to the body 12, with both being configured in overlying relation with the first and second ports 34, 36, wherein the first and second rupturable members 38, 40 can be formed as a single piece of material, such as being formed by the material of the bottom layer 26. The first and second rupturable members 38, 40 are rupturable, such as via the puncture members 30, by way of example and without limitation, whereupon the ruptured first and second rupturable members 38, 40 are provided with the openings 32 to bring the first and second channels into fluid communication with the chamber 20 through the first and second ports 34, 36.

Upon selectively piercing at least one of the first and second rupturable members 38, 40 and forming the opening(s) 32 therein, the desired reagent 42 can be introduced through a corresponding one of the channels having an opened port, shown, by way of example and without limitation as being introduced through the first channel 14, through the first port 34 and into the chamber 20. With the reagent 42 being introduced into the chamber 20, the reagent 42 and the reactant 22 can be mixed together to initiate the desired assay within the chamber 20.

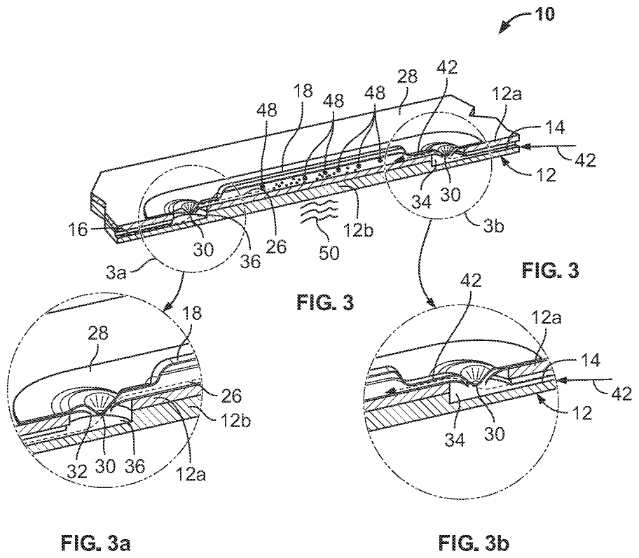

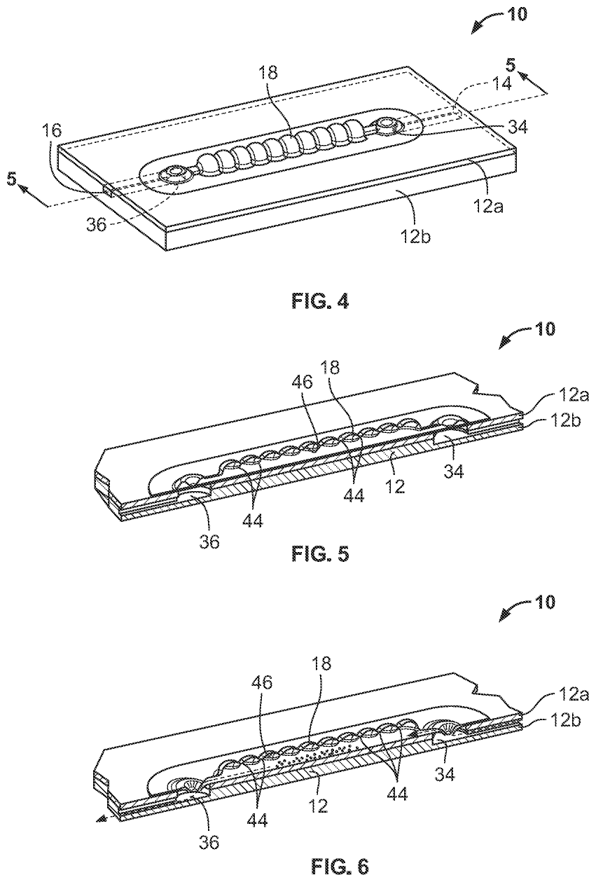

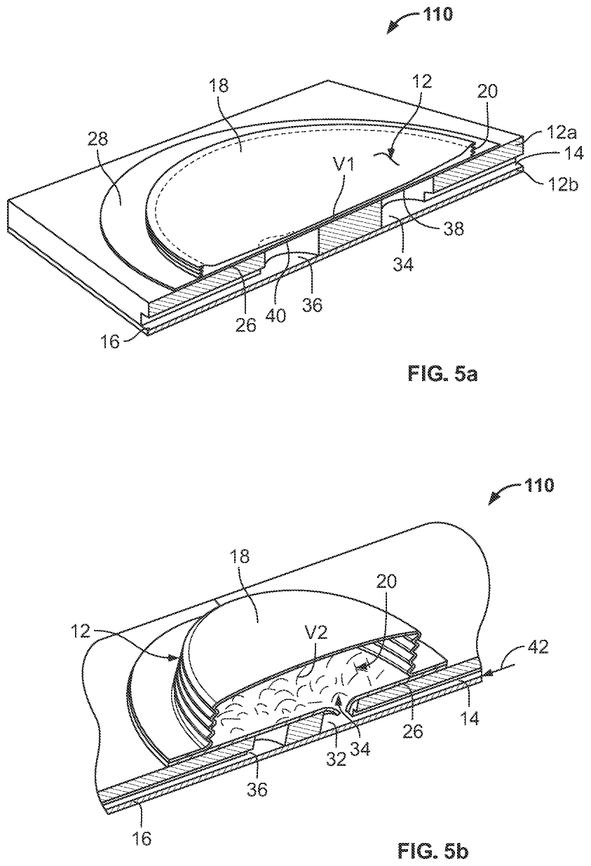

To facilitate mixing the reagent 42 homogenously with the reactant 22, an agitation mechanism 44 can be disposed within the chamber. The agitation mechanism 44 is configured to agitate the flow of a media, herein the reagent 42, entering the chamber 20 to quickly and thoroughly mix the reagent 42 with the reactant 22. The agitation mechanism 44 tends to establish a non-laminar flow of the reagent 42 as the reagent 42 flows against and adjacent the agitation mechanism 44. The agitation mechanism 44 can be formed on an inner surface 46 (FIGS. 5 and 6) of the shroud 18, with the inner surface 46 being directly exposed to the chamber 20. For example, the agitation mechanism 44 can be formed via a plurality of protrusions 44 extending into the chamber 20, with the protrusions 44 being shown forming an undulating, corrugated contour of alternating peaks and valleys in wave-like form. Alternately, the agitation mechanism 44 can be formed by one or more solid members 48 (FIGS. 2 and 3) contained in the chamber 20, with the one or more solid members 48 being free to move within the chamber 20 when at least one of the first and second rupturable members 38, 40 is in the ruptured state to facilitate mixing the reagent 42 with the reactant 22. The one or more solid members 48 can be restrained against movement within the chamber 20 when the first and second rupturable members 38, 40 are in their non-ruptured states. The restraining can be performed by adhering the solid members 48 to a surface within the cavity 20, by way of example and without limitation, with the adherent being dissolvable by the reagent 42 as it enters the cavity 20. Movement of the solid members 48 can be facilitate by gravity simply by tilting the cartridge 10 sufficiently to cause movement of the solid members 48.

In accordance with a further aspect, the agitation mechanism 44 can be formed by ferrous material, such as illustrated with regard to the solid members 48 wherein the solid members 48 can have a ferrous content, contained within the chamber 20. The ferrous material 48 is configured to move within the chamber 20 upon being selectively exposed to an external magnetic field 50. The magnetic field 50 can be located and directed as desired to obtain the desire movement of the ferrous material 48 within the chamber 20.

In FIG. 5a, a disposable diagnostic device 110 in accordance with a further aspect is illustrated, wherein the same reference numerals are used to identify like features. The cartridge 110 includes the shroud 18, which can be provided having a compressed state defining a minimal first volume V1 inside the chamber 20 when the first rupturable member 38 is in its non-ruptured state and when the second rupturable member 40 is in its non-ruptured state, such that the cartridge 110 is in its non-actuated state. The shroud 18 can further be formed to attain an unfolded, expanded state defining a second volume V2 inside the chamber 20 when at least one of the first rupturable member 38 is in its ruptured state and/or when the second rupturable member 40 is in its ruptured state, shown in FIG. 5b as having the first rupturable member 38 being ruptured to allow the ingress (inflow) of the reagent 42 through the first channel 14 and into the cavity 20, with the second volume V2 being greater than said first volume V1. Accordingly, the expanded state results from the ingress of the reagent 42 into the cavity 20 causing the shroud 18 and the underlying cavity to expand. Then, upon expansion of the cavity 20, with the reagent 42 and the reactant 22 mixed together within the cavity 20, the remaining first or second rupturable member 38, 40 can be opened to allow the mixture to be expelled from the cavity 20 through the desired first or second channel 14, 16 to continue the assay, as desired.

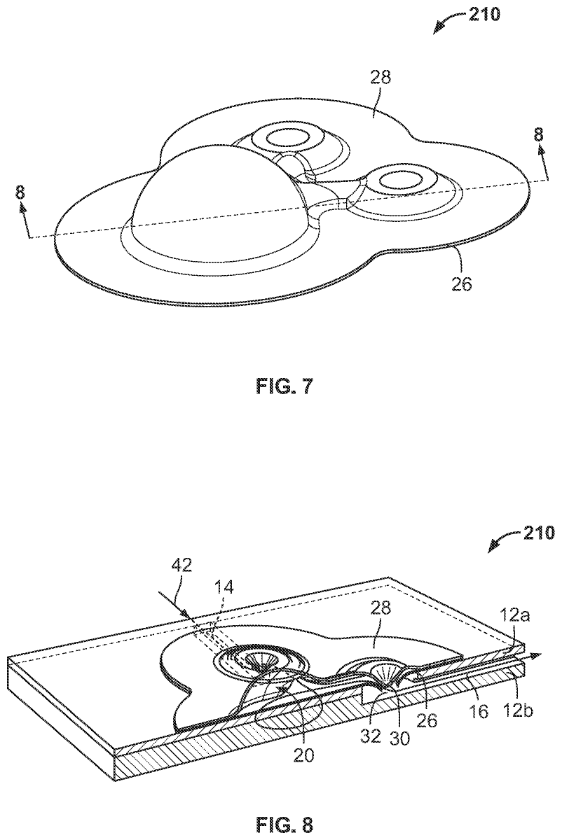

In FIGS. 7 and 8, a disposable diagnostic device 210 in accordance with a further aspect of the invention is illustrated, wherein the cartridge 210 has similar features identified by like reference numerals in FIG. 8. Further discussion is believed unnecessary in view of the discussion above for such enumerated features.

The foregoing description of the embodiments has been provided for purposes of illustration and description. It is not intended to be exhaustive or to limit the disclosure or claims. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure and claims, wherein the claims ultimately define the scope of the invention.

What is claimed is:

1. A disposable diagnostic device, comprising:
a body having a first channel and a second channel spaced from said first channel;
a shroud operably fixed to said body, said shroud enclosing a chamber, said chamber being configured in hermetically sealed-off relation from said first channel and said second channel when said disposable diagnostic device is in a non-activated first state and said chamber being in open communication with at least one of said first channel and said second channel when said disposable diagnostic device is in an activated second state;
a reactant disposed in said chamber; and
an inert gas disposed in said chamber, said inert gas protecting said reactant from being exposed to contaminants while in said chamber when said disposable diagnostic device is in said non-activated first state.

2. The disposable diagnostic device of claim 1, wherein said first channel is sealed-off from said chamber by a first rupturable member covering a first port when said first rupturable member is in a non-ruptured state and said first channel is in fluid communication with said chamber through said first port when said first rupturable member is in a ruptured state, said second channel is sealed-off from said chamber by a second rupturable member covering a second port when said second rupturable member is in a non-ruptured state and said second channel is in fluid communication with said chamber through said second port when said second rupturable member is in a ruptured state.

3. The disposable diagnostic device of claim 2, wherein said first rupturable member is fixed to said body and wherein said second rupturable member is fixed to said body.

4. The disposable diagnostic device of claim 3, wherein said first rupturable member and said second rupturable member are a single piece of material.

5. The disposable diagnostic device of claim 2, further including an agitation mechanism disposed within said chamber, said agitation mechanism being configured to agitate the flow of a media entering said chamber from one of said first channel through said first port and said second channel through said second port to mix said media with said reactant.

6. The disposable diagnostic device of claim 5, wherein said agitation mechanism is formed on an inner surface of said shroud, said inner surface being exposed to said chamber.

7. The disposable diagnostic device of claim 6, wherein said inner surface has a plurality of protrusions extending into said chamber to form said agitation mechanism.

8. The disposable diagnostic device of claim 5, wherein said agitation mechanism is formed by one or more solid members contained in said chamber, said one or more solid members being free to move within said chamber when at least one of said first and second rupturable members is in said ruptured state to mix said media with said reactant.

9. The disposable diagnostic device of claim 8, wherein said one or more solid members is restrained against movement within said chamber when said first and second rupturable members are in said non-ruptured states.

10. The disposable diagnostic device of claim 5, wherein said agitation mechanism is formed by ferrous material contained within said chamber, said ferrous material being configured to move within said chamber upon being selectively exposed to an external magnetic field.

11. The disposable diagnostic device of claim 2, wherein said shroud is formed of a compliant material configured to be depressed and substantially flattened to motivate a flow of a media entering said chamber from one of said first channel through said first port and from said second channel through said second port and out the other of said first port through said first channel and out said second port through said second channel.

12. The disposable diagnostic device of claim 11, wherein said shroud has a compressed state defining a first volume inside said chamber when said first rupturable member is in its non-ruptured state and when said second rupturable member is in its non-ruptured state, and an expanded state defining a second volume inside said chamber when at least one of said first rupturable member is in its ruptured state and when said second rupturable member is in its ruptured state, said second volume being greater than said first volume.

13. A method of constructing a disposable diagnostic device, comprising:
providing a diagnostic cartridge body having a plurality of microfluidic channels;
providing a shroud;
disposing a reactant between the shroud and the diagnostic cartridge body;
fixing the shroud to the diagnostic cartridge body to seal-off a chamber between the shroud and the diagnostic cartridge body, wherein the reactant is contained in the chamber and the chamber is configured for selective fluid communication with the plurality of microfluidic channels; and
performing the disposing and fixing steps in a vacuum atmosphere.

14. The method of claim 14, further including providing an inert gas in the vacuum atmosphere and sealing some of the inert gas in the chamber with the reactant.

15. The method of claim 14, wherein the fixing step is not performed in a dry-room atmosphere.

16. The method of claim 14, further including providing an agitation mechanism in the chamber to facilitate mixing the reactant with a reagent.
